(19) United States
(12) Patent Application Publication
(10) Pub. No.: US 2022/0117158 A1
(43) Pub. Date: Apr. 21, 2022

(54) METHOD AND APPARATUS FOR DETERMINING AND MAPPING CROP HEIGHT

(71) Applicant: Topcon Positioning Systems, Inc., Livermore, CA (US)

(72) Inventor: Marko LAMPRECHT, Gera (DE)

(73) Assignee: Topcon Positioning Systems, Inc., Livermore, CA (US)

(21) Appl. No.: 17/071,997

(22) Filed: Oct. 15, 2020

(51) Int. Cl. A01D 41/127; A01D 34/14; A01D 57/12; A01B 79/02; G01B 17/02; G01B 11/24; G01C 9/02; G01B 7/30; G01S 19/01; G01N 33/00

(52) U.S. Cl. CPC A01D 41/127; A01D 34/14; A01D 57/12; A01B 79/02; G01N 33/0098; G01B 11/24; G01C 9/02; G01B 7/30; G01S 19/01; G01B 17/02

(57) ABSTRACT

A method for mapping a height of a crop in a field divided into a plurality of areas includes determining a height of a cutting bar of an agricultural machine and receiving data from a crop height sensor. The height of crops sensed by the crop height sensor is determined based on the height of the cutting bar and data from the crop height sensor. The crop height is then associated with one of a plurality of areas of the field based on a location of the crop height sensor. In one embodiment, the height of a reel of the agricultural machine is also used in determining the height of crops. The crop height data is used to generate a field map that is used to generate a field treatment plan.

### FIELD OF THE INVENTION

The present disclosure relates generally to agricultural operations, and more particularly to mapping crop heights in a field.

### BACKGROUND

Agricultural fields need to be utilized efficiently since they are a limited resource of finite size. Agricultural fields are typically utilized to produce a maximum income per area. Specific treatment of the crops is required to produce the maximum economic yield. Treatment of the crops typically consists of applying pesticide, fertilizing, and watering in amounts to promote a desired growth of the crops. Incorrect treatment of crops can result in low growth which reduces the maximum economic yield. Incorrect treatment of crops can also result in the crops growing too large. When certain crops, such as wheat, grow too large, the stem of the plant cannot support the weight of the seed and the plant experiences lodging. Many crops also experience lodging when infected with pests. Such lodging due to pests occurs when pests infect a base region of the stem of a plant. Lodging is a condition of a plant in which the plant falls over due to the excessive weight of the seed located near the top of the stem of the plant in relation to the strength of the stem. Lodging has an adverse impact for a variety of reasons. Lodging reduces the maximum economic yield because it causes harvesting to be less efficient. Lodging can cause a slower harvest, higher fuel consumption, smaller grain (less yield), grain loss due to grain remaining on the ground, increased risk of damage to harvesting equipment by stones and foreign objects, spoilage of grain, rotten grain, mycosis and the toxic substances that mycosis produces, and the cost of drying grain that has become moist from ground water. What is needed is a method to determine an ideal treatment plan for a crop so that the crop grows in a manner to produce the maximum economic yield.

### SUMMARY

A method for mapping a height of a crop in a field divided into a plurality of areas includes determining a height of a cutting bar of an agricultural machine and receiving data from a crop height sensor. The height of crops sensed by the crop height sensor is determined based on the height of the cutting bar and data from the crop height sensor. The crop height is then associated with one of a plurality of areas of the field based on a location of the crop height sensor. In one embodiment, the height of a reel of the agricultural machine is also used in determining the height of crops. Data from a conveyor inclinometer along with a known height of a rotation axis associated with the conveyor inclinometer are used to determine a height of the cutting bar. Data from a reel inclinometer along with a height of a rotation axis associated with the reel are used to determine a height of the reel. In one embodiment, crop height data is used to generate a field map that is used to generate a field treatment plan. In one embodiment, a size of seed harvested are determined using a seed size sensor and the field treatment plan is further based on the size of seeds. The field treatment plan, in one embodiment, comprises one of land levelling, altered tillage, adopted seed rate, adopted seed variety, span over weeding, fertilizer application, fertilizer application, pesticide application, growth regulator application, and irrigation. Field treatment plans can be improved if the result of the plan is checked and compared with prior field treatment plans for a particular area.

### BRIEF DESCRIPTION OF THE DRAWINGS

FIG. 1A depicts an upright plant;

FIG. 1B depicts a lodging plant;

FIG. 2A depicts a cutting height for an upright plant;

FIG. 2B depicts a cutting height for a lodging plant;

FIG. 3A depicts a combine positioned to harvest a lodging crop;

FIG. 3B depicts a combine positioned to harvest an upright crop;

FIG. 4A depicts a reel positioned to harvest an upright crop;

FIG. 4B depicts a reel positioned to harvest a lodging crop;

FIG. 5A depicts a reel position with respect to a cutting bar to harvest an upright crop;

FIG. 5B depicts a reel position with respect to a cutting bar to harvest a lodging crop;

FIG. 6A depicts components of a combine for determining a height of header elements and a reel;

FIG. 6B depicts components of a combine for determining a height of header elements and a reel;

FIG. 7 depicts a side view of components of a combine for determining crop height;

FIG. 8 depicts a front view of components of a combine for determining crop height;

FIG. 9 depicts a controller and related components for sensing combine parameters and crop parameters;

FIG. 10 depicts a field in which a combine is harvesting a crop; and

FIG. 11 depicts a flow chart of a method according to one embodiment.

### DETAILED DESCRIPTION

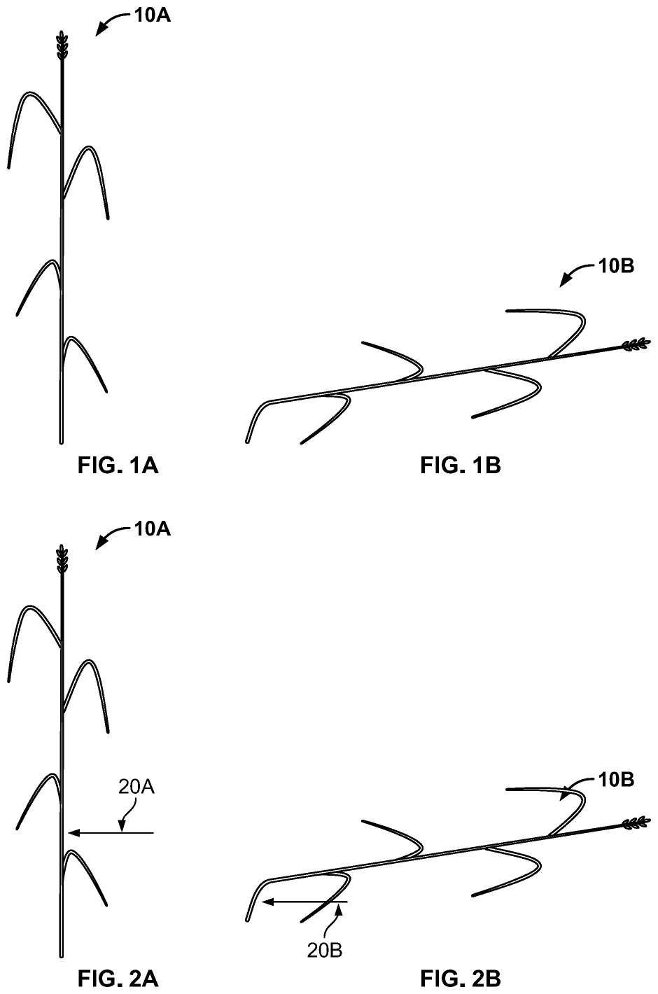

FIG. 1A depicts a healthy and fully grown plant 10A, specifically a wheat plant, standing upright. FIG. 1B depicts lodging plant 10B that has fallen over. Lodging is the falling over of a plant. Lodging can occur for a variety of reasons including crops being overfed or pest infestation. Overfeeding of a plant can cause the seeds located at the top of the plant to grow large and heavy. The weight of seeds overcomes the ability of the plant to remain upright. Pest infestation can weaken the stem of a plant and reduce the stem's ability to maintain an upright orientation. Lodging affects the method used to harvest plants.

FIG. 2A depicts healthy and fully grown plant 10A with arrow 20A indicating where plant 10A should be cut for harvesting. FIG. 2B depicts lodging plant 10B with arrow 20B indicating where plant 10B should be cut for harvesting. As shown in FIG. 2B, the cutting height required for plant 10B is lower than the cutting height required for plant 10A.

Lodging crop lays dense with less air flow through the crop, causing slower drying after rain and dew and catching more water evaporated from the ground and wetting the grain itself. Longer wet periods can cause the grain to spoil or germinate. Spoiled grain is not usable for food or even feeding. Grain that started to germinate can't be used as seed or in malt production. A header of a combine needs to be lowered in order to harvest lodging crops which increases the risk of the combine collecting earth and stones which can damage the combine. The grain flow through the combine needs to pass small openings for proper processing. Earth and stones collected due to a lowered header can damage these small passages and cause machine down time and repair cost.

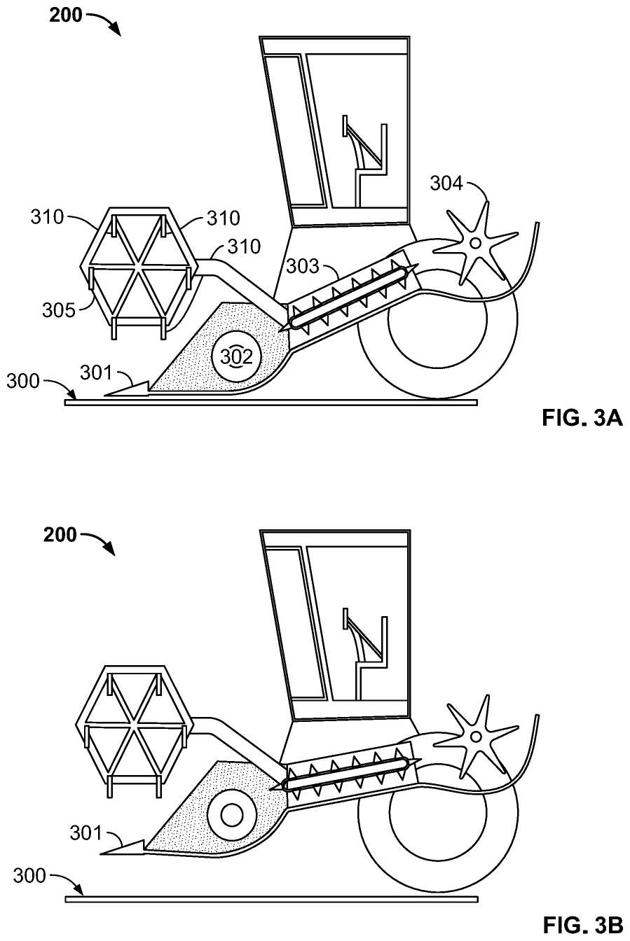

FIG. 3A depicts components of combine 200 oriented in a position for cutting lodged crops. Cutting bar 301 is oriented near ground 300 in order to harvest lodged crop, such as plant 10B (shown in FIG. 1B). Reel 305 rotates counter-clockwise (as viewed from the left side of the combine as shown in FIG. 3A) and urges the top portion of plants being harvested toward auger 302. Auger 302, in one embodiment, is a screw like component that urges harvested plants toward grain conveyor 303. Grain conveyor 303 moves harvested plants toward threshing drum 304. Threshing drum rotates and mechanically separates the seeds of the harvested plants from the stems of the harvested plants.

FIG. 3B depicts cutting bar 301 located in a position for cutting upright crops. Cutting bar 301 is located at a height above ground 300 to cut the stem of plants of a crop at a height identified by arrow 20A of a FIG. 2A.

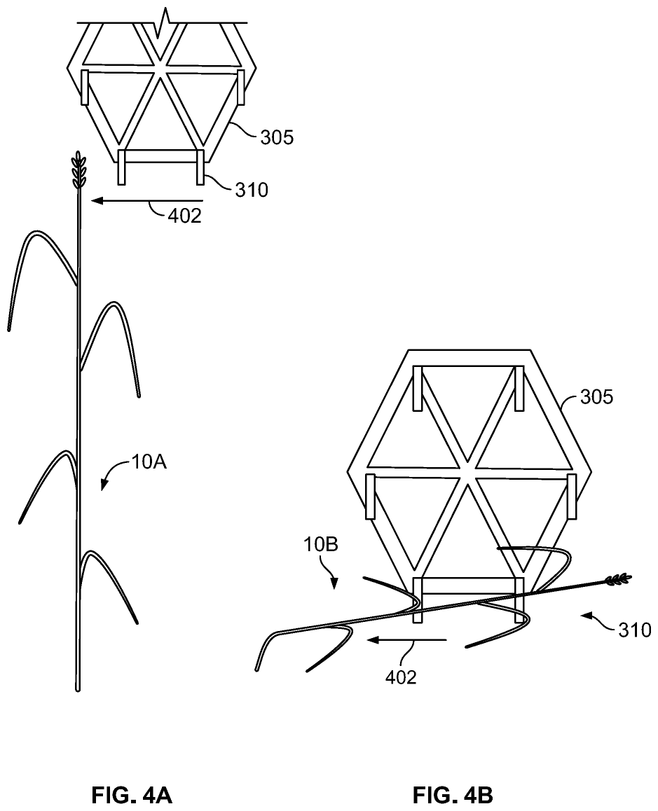

FIG. 4A depicts a location of reel 305 with respect to upright plant 10A. Reel 305 is located with respect to plant 10A at a height so that tines 310 collide with a top section of plant 10A where seeds of the plant are located. Tines 310 collide with the top section of plant 10A as the combine moves in the direction indicated by arrow 402 (toward plant 10A) and reel 305 rotates counter-clockwise.

FIG. 4B depicts a location of reel 305 with respect to lodged plant 10B. Reel 305 is located with respect to plant 10B at a height so that tines 310 collide with a top and middle section of plant 10B. Tines 310 collide with the top and middle section of plant 10B as the combine moved in the direction indicated by arrow 402 (toward plant 10B) and reel 305 rotates counter-clockwise.

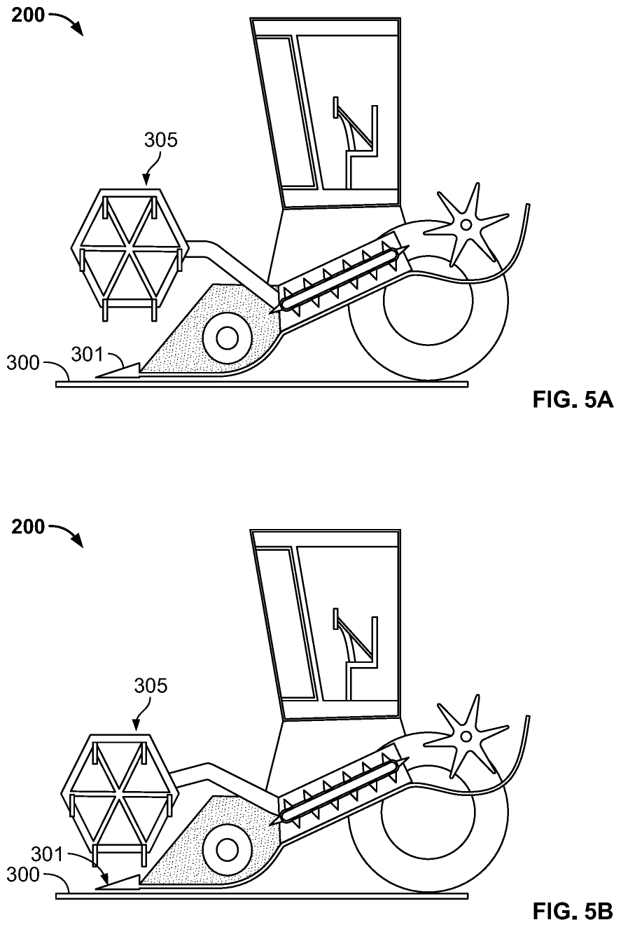

FIG. 5A depicts a position of reel 305 with respect to cutting bar 301. FIG. 5A depicts reel 305 located a distance from cutting bar 301 thereby positioning reel 305 to collide with the top section of an upright plant (such as plant 10A shown in FIG. 1A) being harvested.

FIG. 5B depicts a position of reel 305 with respect to cutting bar 301. FIG. 5B depicts reel 305 located a distance from cutting bar 301 thereby positioning reel 305 to collide with a top and middle section of lodged plant (such as plant 10B shown in FIG. 1B).

It should be noted that FIGS. 5A and 5B illustrate the position of reel 305 with respect to cutting bar 301. The height of cutting bar 301 shown in FIGS. 5A and 5B is not indicative of the height of cutting bar 301 required to cut crops.

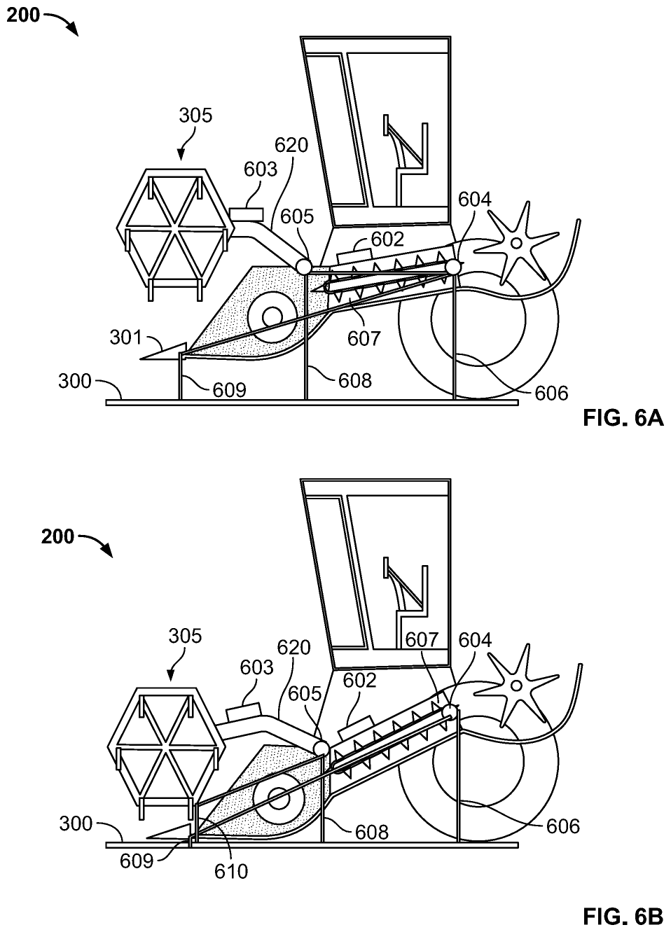

FIG. 6A depicts components of combine 200 and sensors used to determine the positions of the components. Conveyor inclinometer 602 is a sensor for determining an inclination of grain conveyor 303. Conveyor inclinometer 602 can be any type of sensor that can sense an angle relative a predetermined axis such as a sensor that can sense an angle relative to the direction of the gravity vector or a potentiometer that can measure an angle with respect to the predetermined axis. As grain conveyor 607 is moved about conveyor rotation axis 604, conveyor inclinometer 602 determines the inclination of grain conveyor 303. Reel inclinometer 603 is a sensor for determining an inclination of reel member 620 to which reel 305 is attached. As reel 305 is moved about rotation axis 605, reel inclinometer 603 determines the inclination of reel member 620 to which reel 305 is attached. Data from conveyor inclinometer 602 and reel inclinometer 603 and be used to determine the position of cutting bar 301 and reel 305.

Cutting bar height 609 above ground 300, as shown in FIG. 6A can be determined as follows. Conveyor rotation axis height 606 is a known, and typically fixed, height above ground 300. Conveyor rotation axis height 606 and an inclination of conveyor 607 determined by conveyor inclinometer 602 can be used to determine rotation axis height 608. The cutting bar height 609 can be determined based on a known spatial relationship between rotation axis 605 and cutting bar 301.

Reel height 610 above ground 300, as shown in FIG. 6B can be determined as follows. Conveyor rotation axis height 606 is a known, and typically fixed, height above ground 300. Conveyor rotation axis height 606 and an inclination of conveyor 607 determined by conveyor inclinometer 602 can be used to determine rotation axis height 608. Rotation axis height 608 and an inclination of reel member 620 supporting reel 305 determined by reel inclinometer 603 can be used to determine reel height 610 above ground 300. In one embodiment, the determination of reel height 610 is also based on a known spatial relationship between reel 305 and rotation axis 605.

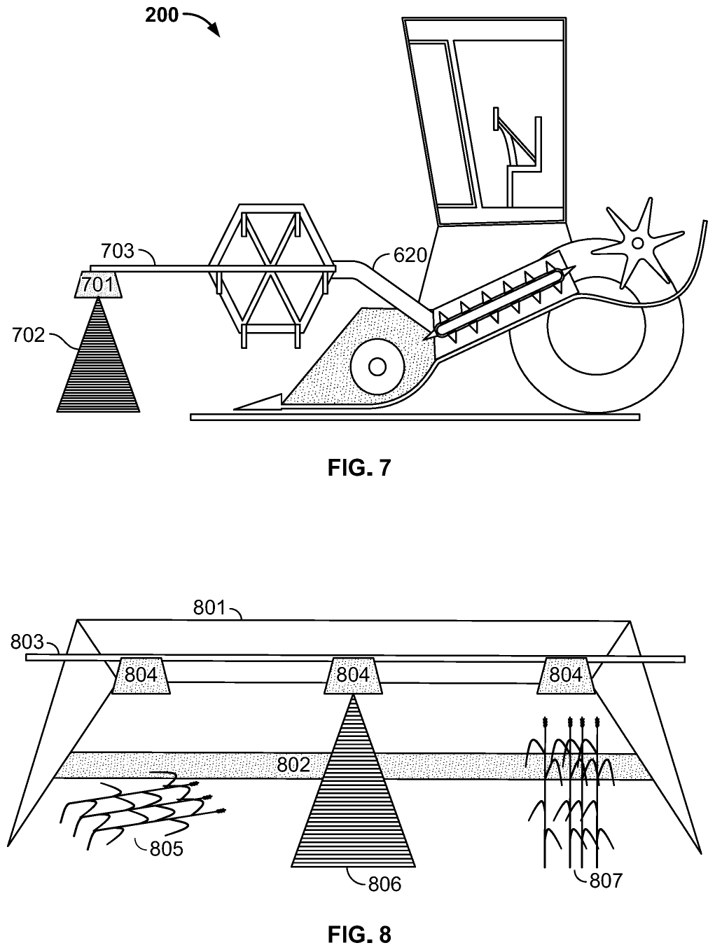

FIG. 7 depicts a side view of a combine having sensor 701 for detecting plant height. Sensor 701 is mounted to sensor bracket 703 which is attached to reel member 620. Sensor 701 detects the height of crops located within sensor scanning region 702. Sensor 701, in one embodiment, is a sonic sensor but can be other types of sensors such as laser, LIDAR, and/or optical sensors.

FIG. 8 depicts a front view of combine 200 having crop height sensors 804 attached to sensor bracket 803. As shown in FIG. 8, sensors 804 are spaced along sensor bracket 803 to cover a desired portion of crops that will be cut by cutting bar 802 attached to a lower portion of header 801. Although three sensors 804 are shown in FIG. 8, more or less sensors 804 can be used depending on a desired granularity of data with respect to a size of an area. Each of sensors 804 has an associated scanning region 806. It should be noted that scanning region 806 associated with sensor 804 located approximately in the center of sensor bracket 803. The sensor regions associated with sensors 804 located closer to the ends of sensor bracket 803 are omitted for clarity. Sensor 804 shown on the left side of FIG. 8 is depicted scanning lodged crops 805 while sensor 804 shown on the right side of FIG. 8 is depicted scanning upright crops 807.

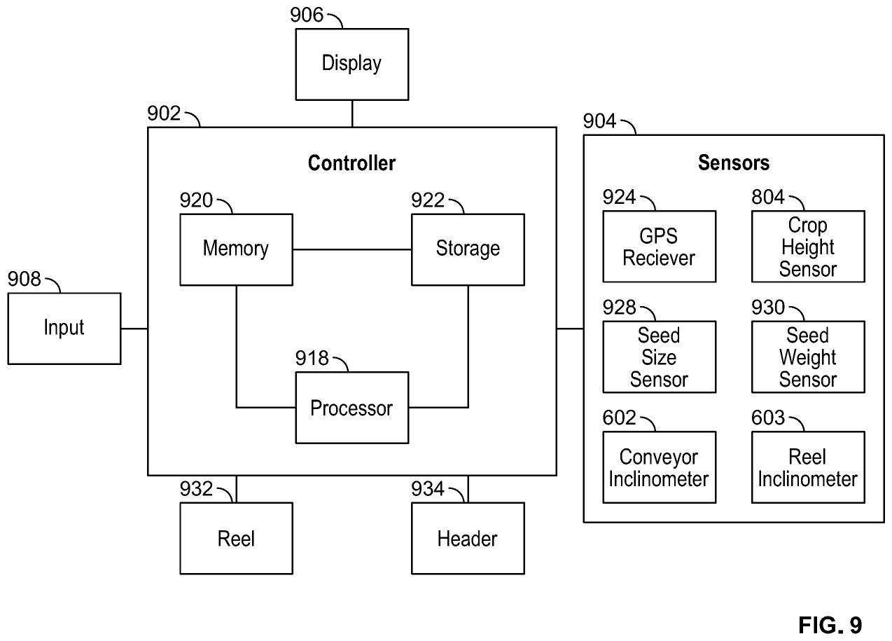

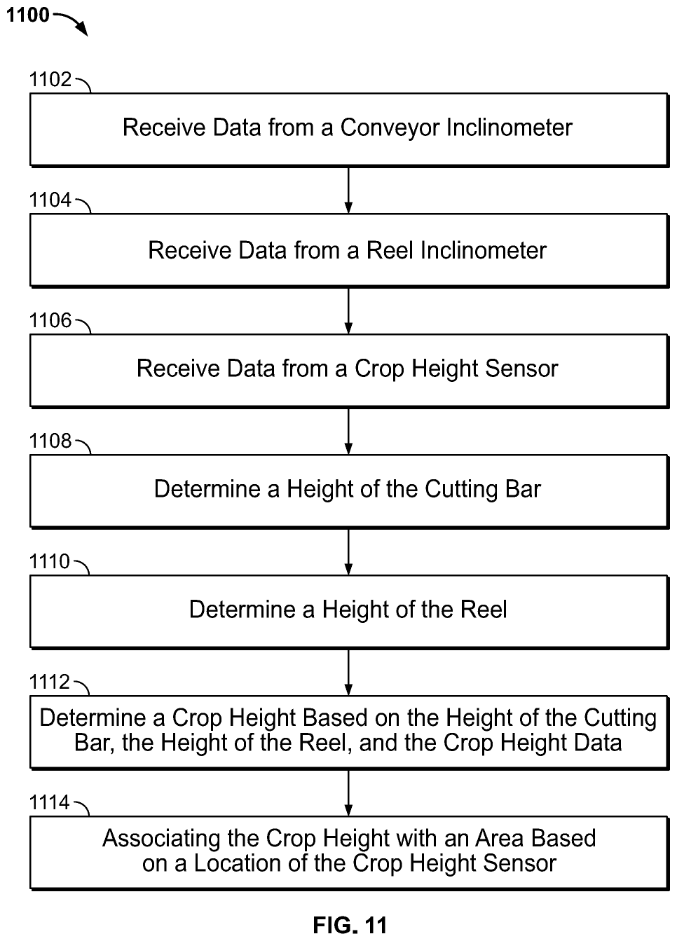

FIG. 9 depicts a schematic of components of combine 200 related to sensing crop height and mapping crop height according to an embodiment. Controller 902, in one embodiment, is implemented using a computer. Controller 902 contains a processor 918 which controls the overall operation of the controller 902 by executing computer program instructions which define such operation. The computer program instructions may be stored in a storage device 922, or other computer readable medium (e.g., magnetic disk, CD ROM, flash drive, cloud drive, etc.), and loaded into memory 920 when execution of the computer program instructions is desired. Thus, the method steps of FIG. 11 (described below) can be defined by the computer program instructions stored in the memory 920 and/or storage 922 and controlled by the processor 918 executing the computer program instructions. For example, the computer program instructions can be implemented as computer executable code programmed by one skilled in the art to perform an algorithm defined by the method steps of FIG. 11. Accordingly, by executing the computer program instructions, the processor 918 executes an algorithm defined by the method steps of FIG. 11. One skilled in the art will recognize that an implementation of a controller could contain other components as well, and that controller 902 is a high level representation of some of the components of such a controller for illustrative purposes.

Combine 200 also includes sensors 904 for determining a location of the agricultural machine and various parameters of crops. In one embodiment, the location of combine 200 is determined using GPS receiver 924 and/or an inertial measurement unit (IMU). Sensors 904 also include crop height sensor 804 (shown in FIGS. 7 and 8) for detecting a height of crops prior to cutting and processing by combine 200. Crop height sensor 804, in one embodiment is an analog sensor that can detect a height of crops located near the sensor. Sensors 904 also include seed size sensor 928 for generating data pertaining to a size of seeds harvested by combine 200. Seed size sensor 928, in one embodiment, is an optical sensor for detecting a size of seed harvested by combine 200. Seed size sensor 928 can be located in any location of combine 200 where seed with seed husk removed is moved while processed. For example, seed size sensor 928 can be located downstream of a threshing drum and separator. In one embodiment, seed size sensor 928 can be at the bottom of an auger for moving the seeds. Sensors 904 also include a weight sensor 930 for determining a weight of seeds harvested by combine 200. Weight sensor 930 can be any type of sensor that can measure weight directly, such as a load cell. Weight sensor 930 can also be a sensor that measures weight indirectly, such as a volumetric sensor or force sensor. Since harvested crops are moved through combine 200 as they are processed, weight sensor 930 can alternatively be located in other locations of combine 200 where processing of crops occurs. For example, weight sensor 930 can be located on an auger or elevator that transports that seeds.

Sensors 904 also include conveyor inclinometer 602 and reel inclinometer 603). Sensors 904, in one embodiment, can include additional sensors (not shown) such as a camera, infrared scanner, or other types of devices for determining parameters of crops in a field in which the agricultural machine is located. Sensors 904, in one embodiment, can also include various sensors such as temperature and pressure sensors associated with various components of the agricultural machine in order to monitor a state of combine 200.

Input 908, in one embodiment, includes inputs from a user operating combine 200. In one embodiment, input 908 can include one or more components for controlling movement of combine 200. For example, a steering wheel, gas pedal and brake pedal can be used to drive the agricultural machine along a desired path. Input 908 can also include various buttons, levers, and switches for controlling operation of reel 305, header 801, and other components of the agricultural machine. Input 908 can also include inputs from a user via input devices such as touch screens and other types of inputs.

Display 906, in one embodiment, is located in the cab of combine 200 and displays information to a user. Display 906 can be any type of display such as a touch screen, a light emitting diode display, a liquid crystal display, heads-up projected display, etc. Display 906 presents various information to a user concerning combine 200, a field, etc. In one embodiment, a display is not used and data concerning a crop is captured and then transferred to another device, such as a desktop computer, for analysis.

Controller 902 is also in communication with reel 932 which, in one embodiment, is a device for controlling the height of reel 932. In one embodiment, reel 932 is controlled by a user and controller 902 senses various parameters of the operation of reel 305 such as rotation speed. In one embodiment, user inputs received via input 908 are received by controller 902 and used to command reel 305 to operate in response to the user inputs.

Controller 902 is also connected to header 934 which, in one embodiment, is a device for controlling the height of header 801 to which cutting bar 301 is attached. As such, the height of header 801 is related to the height of cutting bar 301. In one embodiment, header 801 is controlled by a user and controller 902 senses various parameters of the operation of header 801 such as vertical movement. In one embodiment, user inputs received via input 908 are received by controller 902 and used to command header 801 to operate in response to the user inputs.

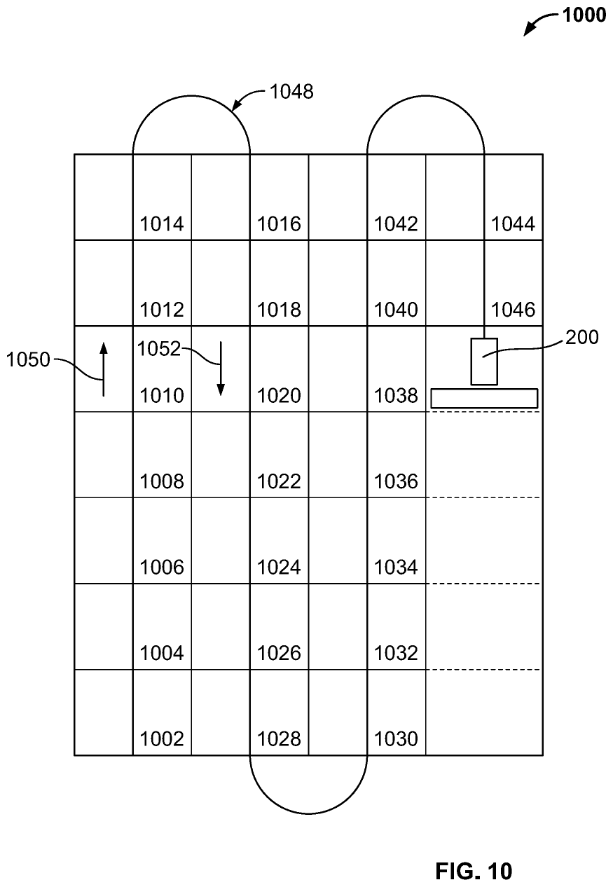

FIG. 10 depicts combine 200 in the process of harvesting crops from field 1000 according to one embodiment. Field 1000 is shown divided into a plurality of grid elements (also referred to as a plurality of areas) defined by columns and rows according to one embodiment. Combine 200 has traversed field 1000 along path 1048 from grid element 1002 through grid element 1014 in a first direction shown by arrow 1050. Combine 1000 has turned 180 degrees after traversing grid element 1014 in the first direction to traverse field 1000 from grid element 1016 through grid element 1028 in a second direction as shown by arrow 1052. Combine 200 has turned 180 degrees after traversing grid element 1028 to traverse field 1000 from grid element 1030 through grid element 1042 in the first direction. Combine 200 has turned 180 degrees after traversing grid element 1042 to traverse field 1000 through grid element 1044 and grid element 1046 in the second direction. Combine 200 will continue to traverse field 1000 in the second direction from its position shown in FIG. 10.

As combine 200 traverses field 1000, crop height sensor 804 determines the height of crops being harvested in a grid element of field 1000. The particular grid element in which combine 200 is located is determined using GPS receiver 624. In one embodiment, the location of crops detected by crop height sensor 84 is calculated based on the difference between the location of GPS receiver 624 and the location of crop height sensor 804. For example, GPS receiver 624 can be located in an operator cab of combine 200 approximately 10 feet rearward and 4 feet to the right of crop height sensor 804. As such, the location of crops detected by crop height sensor 804 is 10 feet forward and 4 feet to the left of the location of GPS receiver 624. This difference in location can be determined and accounted for in determining the location of crops detected by crop height sensor 804 and the location of GPS receiver 624. In one embodiment, GPS receiver 624 determines a location of an antenna associated with GPS receiver 624. Similar

The data from crop height sensor 804 and GPS receiver 624 are used to generate a map depicting crop heights in various locations of field 1000. As shown in FIG. 10, field 1000 has been divided into a plurality of grid elements. Each element of the grid (e.g., 1002-1046) can be associated with an average crop height determined for that particular element. As such, a crop height map can be generated using the information obtained using GPS receiver 624 and crop height sensor 804. The 4 by 7 grid shown in FIG. 10 is an example. The dimensions of the grid shown in FIG. 10 (i.e., the number of columns and rows used to generate the grid) can be selected based on a desired resolution as well as the size of the field.

In one embodiment, a width of an element of a grid is equal to a width of crop that a combine can harvest in one pass. For example, as shown in FIG. 10, the width of each column is equal to the width of crop combine 200 can harvest as it travels in field 1000. In one embodiment, the width of an element of a grid is based on a width of a scanning region (e.g., scanning region 806 shown in FIG. 8). For example, when multiple crop height sensors are used, combine 200 collects data from each of a multiple crop height sensors to generate data associated with grid elements each having a width less than the width of crop that a combine harvests in a single pass.

In one embodiment, the shape of each grid element (or area) can be rectangular, triangular, hexagonal, polygonal, etc. In one embodiment, small areas or points can be used to represent areas forming a density map.

In one embodiment, additional sensors may be used to acquire data relating to various parameters. For example, seed weight can be sampled using a light beam that seeds travel past, such as when seeds are being moved through combine 200 after the crop has been threshed. Alternatively, the weight of seeds can be measured using a force detection device, such as a load cell. Seed weight can measured together with the grain moisture. True yield (i.e., the true weight of seeds) can be determined if the moisture content of seeds can be determined. For example, wheat has a storage moisture of 14%. This is the level it can safely be stored and it is also used for calculating the monetary amount for which seed will be bought or sold. If seeds are harvested in bad conditions, the moisture can be higher. This higher moisture content can cause incorrect calculations of yield which can lead to inaccurate cost estimates. A moisture sensor can be used to determine moisture content of seeds. Moisture sensors can incorporate temperature sensors to allow for compensation of measurement errors caused by temperature of seed.

The generated crop height map is used, in one embodiment, to determine a field treatment plan for future plantings in the same field. For example, combine 200 traverses field 1000 harvesting crops and gathering data pertaining to crop height, crop weight, and seed size for each grid element as the crops in each grid element are harvested. The gathered data is then used to generate a crop height map. The crop height map and data pertaining to crop weight and seed size of crops harvested from each grid element are then analyzed to determine if crops in each grid element were overfed or underfed. In one embodiment, soil samples from each grid element can also be obtained and analyzed. The analyzed soil samples can be considered with the other crop parameters described above in generating of the crop treatment plan. A field treatment plan for a future planting can be generated for each grid element based on the crop height, crop weight, and seed size determined for each grid element.

In one embodiment, a field treatment plan is generated for a particular grid element when data for that particular grid element is available. For example, a field treatment plan can be generated for a particular grid element immediately after the data for the grid element is acquired. In one embodiment, a field treatment plan for each grid element of a field is generated after data from all grid elements of the field have been collected. In one embodiment, crop heights of grid elements are compared to one another in order to determine a field treatment plan. It should be noted that a current planting that is being harvested can be referred to as a first planting and a future planting can be referred to as a second planting.

FIG. 11 depicts a flow chart of a method 1100 for mapping crop heights in a field. At step 1102, controller 902 receives data from conveyor inclinometer 602. At step 1104, controller 902 received data from reel inclinometer 603. At step 1106, controller 902 receives data from crop height sensor 804. At step 1108, a height of cutting bar 301 is determined based on conveyor rotation axis height 606 and an inclination of conveyor 607 determined based on data from conveyor inclinometer 602 received at step 1102. At step 1110, a height of reel 305 is determined based on rotation axis height 608 and an inclination of reel member 620 based on data from reel inclinometer 603 received at step 1104. At step 1112, a crop height is determined based on the height of cutting bar 301, the height of reel 305, and crop height data received from crop height sensor 804. At step 1114, the crop height is associated with an area based on a location of crop height sensor 804 when crop height was sensed. Steps 1102 through 1114 are repeated as combine 200 traverses a field (e.g. as shown in FIG. 10) in order to generate a crop height map of a field (e.g. field 1000 shown in FIG. 10).

It should be noted that crop height can be determined based on various factors. For example, crop height can be determined based on cutting bar height alone. However, crop height determined using only the cutting bar height is not accurate enough for some applications. Crop height can also be determined using cutting bar height and data from a crop height sensor. Crop height determined using cutting bar height and data from a crop height sensor is more accurate than crop height determinations using cutting bar height alone. Crop height can also be determined using cutting bar height, data from a crop height sensor and reel height. Crop height determinations using all three parameters are typically the most accurate of the three determinations. It should be noted that cutting bar height, crop height data from a crop height sensor, and reel height can be used individual, or in any combination, to determine crop height.

In one embodiment, controller 902 determines if a crop of the particular grid element was overfed or underfed. In one embodiment, information pertaining to the particular grid element is analyzed to determine if the crops in the particular grid element were overfed or underfed. In one embodiment, the height of the crops, weight of agricultural material harvested, and seed size are used to determine if a crop was overfed or underfed. It should be noted that well fed crops may have high weight and large grains, but if the seeds grow too large and are laying down, the last photosynthesis period is not optimal and the grain filling will decrease causing smaller grain and less weight again. Determining whether a crop has been overfed or underfed can require additional seed and/or crop parameters to be taken into account.

In one embodiment, crop height information a field treatment plan for a future planting of the particular grid element is determined. In one embodiment, the field treatment plan is determined based on whether the crop of the particular grid element was determined to have been overfed or underfed. For example, if a crop height was low and a seed size and weight of agricultural material harvested was high for a grid element, the amount of fertilizer applied to the grid element for a future planting may be reduced. Alternatively, if a crop height was low and a seed size and weight of agricultural material harvested was low for a grid element, the amount of fertilizer applied to the particular grid element for a future planting may be increased. In one embodiment, the treatment plan can include recommendations for both a fertilization schedule and a watering schedule of a particular grid element or adopting the application of other agricultural materials such as a growth regulator. In addition, the application of agricultural materials can be increased or decreased. Each schedule identifies when fertilizer, water, and agricultural materials should be applied to crops of a particular grid element.

In one embodiment, a field treatment plan for a future planting in a particular grid element can be generated based on a prior treatment plan for that particular grid element. For example, if a prior particular treatment plan resulted in overfed crops, the prior particular treatment plan can be used as a baseline for generating a treatment plan for a future planting by reducing the fertilization and watering amounts of the particular treatment plan that resulted in overfed crops. Similarly, if a prior particular treatment plan resulted in underfed crops, the prior particular treatment plan can be used as a baseline for generating a new treatment plan for a further planning by increasing the fertilization and watering amounts of the particular treatment plan that resulted in underfed crops.

The foregoing Detailed Description is to be understood as being in every respect illustrative and exemplary, but not restrictive, and the scope of the inventive concept disclosed herein is not to be determined from the Detailed Description, but rather from the claims as interpreted according to the full breadth permitted by the patent laws. It is to be understood that the embodiments shown and described herein are only illustrative of the principles of the inventive concept and that various modifications may be implemented by those skilled in the art without departing from the scope and spirit of the inventive concept. Those skilled in the art could implement various other feature combinations without departing from the scope and spirit of the inventive concept.

1. A method for mapping a height of a crop in a field, the field divided into a plurality of areas, the method comprising:
determining a height of a cutting bar of an agricultural machine;
receiving crop height data from a crop height sensor;
determining a crop height based on the height of the cutting bar and the crop height data; and
associating the crop height with one of the plurality of areas based on a location of the crop height sensor.

2. The method of claim 1, further comprising:
determining a height of a reel of the agricultural machine,
wherein the determining the crop height is further based on the height of the reel.

3. The method of claim 1, further comprising:
receiving data from a conveyor inclinometer,
wherein the determining the height of the cutting bar is based on the data from the conveyor inclinometer and a height of a rotation axis about which the conveyor moves.

4. The method of claim 2, further comprising:
receiving data from a reel inclinometer,
wherein the determining the height of the reel is based on the data from the reel inclinometer and a height of a rotation axis about which a member supporting the reel inclinometer rotates.

5. The method of claim 1, further comprising:
generating a field treatment plan based on a field map generated based on the associating.

6. The method of claim 5, further comprising:
determining a size of seeds harvested based on data from a seed size sensor of the agricultural machine,
associating the size of seeds harvested with the one of the plurality of areas based on the location of the crop sensor,
wherein the field treatment plan is further based on the size of seeds.

7. The method of claim 5, wherein the field treatment plan comprises one of land levelling, altered tillage, adopted seed rate, adopted seed variety, span over weeding, fertilizer application, fertilizer application, pesticide application, growth regulator application, and irrigation.

8. An apparatus comprising:
a processor; and
a memory to store computer program instructions, the computer program instructions when executed on the processor cause the processor to perform operations comprising:
determining a height of a cutting bar of an agricultural machine;
receiving crop height data from a crop height sensor;
determining a crop height based on the height of the cutting bar and the crop height data; and
associating the crop height with one of a plurality of areas based on a location of the crop height sensor.

9. The apparatus of claim 8, the operations further comprising:
determining a height of a reel of the agricultural machine,
wherein the determining the crop height is further based on the height of the reel.

10. The apparatus of claim 8, the operations further comprising:
receiving data from a conveyor inclinometer,
wherein the determining the height of the cutting bar is based on the data from the conveyor inclinometer and a height of a rotation axis about which the conveyor moves.

11. The apparatus of claim 9, the operations further comprising:
receiving data from a reel inclinometer,
wherein the determining the height of the reel is based on the data from the reel inclinometer and a height of a rotation axis about which a member supporting the reel inclinometer rotates.

12. The apparatus of claim 8, the operations further comprising:
generating a field treatment plan based on a field map generated based on the associating.

13. The apparatus of claim 12, the operations further comprising:
determining a size of seeds harvested based on data from a seed size sensor of the agricultural machine,
associating the size of seeds harvested with the one of the plurality of areas based on the location of the crop sensor,
wherein the field treatment plan is further based on the size of seeds.

14. The apparatus of claim 12, wherein the field treatment plan comprises one of land levelling, altered tillage, adopted seed rate, adopted seed variety, span over weeding, fertilizer application, fertilizer application, pesticide application, growth regulator application, and irrigation.

15. A combine comprising:
a cutting bar;
a reel;
a conveyor inclinometer;
a reel inclinometer;
a crop height sensor; and
a controller for executing computer program instructions which, when executed by the controller, cause the controller to perform operations comprising:
determining a height of the cutting bar of the combine;
receiving crop height data from the crop height sensor;
determining a crop height based on the height of the cutting bar and the crop height data; and
associating the crop height with one of a plurality of areas based on a location of the crop height sensor.

16. The combine of claim 15, the operations further comprising:
determining a height of the reel of the combine,
wherein the determining the crop height is further based on the height of the reel.

17. The combine of claim 15, the operations further comprising:
receiving data from the conveyor inclinometer,
wherein the determining the height of the cutting bar is based on the data from the conveyor inclinometer and a height of a rotation axis about which the conveyor moves.

18. The combine of claim 16, the operations further comprising:
receiving data from the reel inclinometer,
wherein the determining the height of the reel is based on the data from the reel inclinometer and a height of a rotation axis about which a member supporting the reel inclinometer rotates.

19. The combine of claim 16, the operations further comprising:
generating a field treatment plan based on a field map generated based on the associating.

20. The combine of claim 19, the operations further comprising:
determining a size of seeds harvested based on data from a seed size sensor of the combine,
associating the size of seeds harvested with the one of the plurality of areas based on the location of the crop sensor,
wherein the field treatment plan is further based on the size of seeds.
